(19) United States
(12) Patent Application Publication
(10) Pub. No.: US 2011/0028536 A1
(43) Pub. Date: Feb. 3, 2011

(54) METHODS AND COMPOSITIONS FOR TOPOISOMERASE I MODULATED TUMOR SUPPRESSION

(76) Inventors: Ruth A. Gjerset, San Diego, CA (US); Keya Bandyopadhyay, San Diego, CA (US)

Correspondence Address: FOLEY & LARDNER LLP, P.O. BOX 80278, SAN DIEGO, CA, 92138-0278, US

(21) Appl. No.: 12/377,498

(22) PCT Filed: Aug. 16, 2007
(86) PCT No.: PCT/US07/18387

§ 371 Date: Aug. 6, 2010

Related U.S. Application Data
(60) Provisional application No. 60/822,774, filed on Aug. 18, 2006.

(51) Int. Cl. A61K 31/7088; C12N 5/071; A61K 31/4375; C12Q 1/68; C12N 5/09; A61P 35/00

(52) U.S. Cl. 514/44 R; 435/325; 514/283; 435/6

(57) ABSTRACT

Disclosed herein are methods and compositions for enhancing the sensitivity of cells to the effects of topoisomerase I inhibitors. Also disclosed are methods and compositions for inducing apoptosis and/or growth arrest which may be used for tumor suppression.

### FIELD OF INVENTION

This invention relates to the field of cancer therapy.

### BACKGROUND OF INVENTION

The following discussion of the background of the invention is merely provided to aid the reader in understanding the invention and is not admitted to describe or constitute prior art to the present invention.

Topoisomerase I is a nuclear enzyme that plays an important role in cell proliferation. The enzyme catalyzes the uncoiling of DNA during replication and transcription (Pommier, et al., Biochim Biophys Acta 1998;1400(1-3):83-105; Wang, Annu Rev Biochem 1996;65:635-92).

The activity of topoisomerase I is regulated by phosphorylation. Such phosphorylation occurs primarily on serine residues (Turman, et al., Biochem Med Metab Biol 1993;50(2):210-25; Coderoni, et al., Int J Biochem 1990;22(7):737-46; Kaiserman, et al., Biochemistry 1988;27(9):3216-22; Samuels, et al., J Biol Chem 1992;267(16):l 1156-62) and appears to be necessary for the initial complex formation between the enzyme and the DNA (Coderoni, et al., Int J Biochem 1990;22(7):737-46).

Human cancers are characterized by uncontrolled proliferation of abnormal cells. Topoisomerase I inhibitors have been used as chemotherapeutic agents that interfere with normal DNA replication and cell division. However, some cancers are not sensitive to such topoisomerase I inhibitors.

### SUMMARY OF THE INVENTION

The present invention provides methods (and related compositions) for increasing the sensitivity of cells (e.g., cancer cells) to the activity of topoisomerase I inhibitors. The invention also provides methods for inducing growth arrest and/or apoptosis in cells (e.g., cancer cells). Further, the invention provides methods for determining the sensitivity of a cell (e.g., a cancer cell) to the effects of a topoisomerase I inhibitor.

The invention is based upon the discovery that cells resistant to topoisomerase I inhibitors frequently have a deficiency in topoisomerase I serine phosphorylation, rendering them less sensitive (or insensitive) to the apoptotic effect of topoisomerase I inhibitors. The deficiency in topoisomerase I phosphorylation reduces the ability of topoisomerase Ito bind p14ARF (ARF), an activator protein. Thus, cancer cells can be assessed for their sensitivity to topoisomerase I inhibitors, prior to initiating therapy, by measuring the level of serine phosphorylation of topoisomerase I, its activity, and/or its ability to bind ARF. Likewise, cells can be sensitized to the effects of topoisomerase I inhibitors by increasing the amount of ARF-topoisomerase I complex formation by increasing, for example, the amount of serine phosphorylation of topoisomerase I or by increasing the amount of ARF available for complexation with topoisomerase I.

An additional feature of the invention is the discovery that apoptosis and/or growth arrest may be induced by disrupting ARF-topoisomerase I complex formation. It is believed that free ARF, released from the ARF-topoisomerase I complexes, increases the biological activity of p53 (a known tumor suppressor gene) by sequestering HDM2, a p53 inhibitor.

Accordingly, in one aspect, the invention provides a method for increasing the sensitivity of a cell to a topoisomerase I inhibitor by contacting the cell with an agent that increases the ARF-topoisomerase I complex formation.

In another aspect, the invention provides a method for inducing cell killing, apoptosis, and/or growth arrest in a cell by contacting the cell with an agent that increases ARF-topoisomerase I complex formation, and further contacting the cell with a topoisomerase I inhibitor.

In one embodiment, the agent increases the amount of topoisomerase I serine phosphorylation.

In another embodiment, the agent increases the serine kinase biological activity in the cell. Preferably, the serine kinase biological activity is increased in the nucleus of the cell, the nucleolus, or in the peri-nucleolar region. Suitable agents include, for example, serine kinase agonists, activators, and cofactors. Other agents include vectors encoding a serine kinase enzyme, operably linked to a promoter. Preferably, the serine kinase phosphorylates topoisomerase I on at least one serine residue (e.g., 1, 2, 3, 4, 5, 6, 7, 8, 9, 10, or more serine residues), and wherein such phosphorylation is capable of promoting ARF-topoisomerase I complex formation. Suitable serine kinases include, for example, casein kinase II (CKII) or protein kinase C (PKC).

In other embodiments, the agent is a vector encoding ARF, or a biologically active fragment thereof, operably linked to a promoter. Preferably, the biologically active ARF fragment contains amino acid residues 66-84 of ARF.

In other embodiments, the cell is a cancer cell including, for example, a lung cancer cell, a prostate cancer cell, a hepatocellular carcinoma cell, a breast cancer cell, a colorectal cancer cell, an acute myelogenous leukemia cell, a melanoma cell, an ovarian cancer cell, a neuroendocrine carcinoma cell, a gastric cancer cell, an esophageal cancer cell, a pancreatic cancer cell, and an adenocarcinoma cell. Preferably, the cell is present within a human patient.

In other embodiments, the cell is further contacted with at least one other chemotherapeutic agent. Suitable chemotherapeutic agents include, for example, alkylating agents, anti-metabolites, vinca alkaloikds, and anti-tumor antibodies.

In other embodiments, the topoisomerase I inhibitor stabilizes a topoisomerase I-DNA complex. Preferable topoisomerase I inhibitors include, for example, camptothecin, irinotecan, topotecan, and analogs thereof.

In a related aspect, the invention provides a method for treating cancer in a patient (e.g., a human patient), who has been diagnosed as having cancer, by administering to the patient an agent that increases ARF-topoisomerase I complex formation, and further administering to the patient a topoisomerase I inhibitor. In preferred embodiments, the cancer includes cancer cells which have a reduced level of ARF-topoisomerase I complex formation relative to non-cancerous cells of the same type.

In one embodiment, the agent increases the amount of topoisomerase I serine phosphorylation.

In another embodiment, the agent increases the serine kinase biological activity in the cell. Preferably, the serine kinase biological activity is increased in the nucleus of the cell, the nucleolus, or in the peri-nucleolar region. Suitable agents include, for example, serine kinase agonists, activators, and cofactors. Other agents include vectors encoding a serine kinase enzyme, operably linked to a promoter. Preferably, the serine kinase phosphorylates topoisomerase I on at least one serine residue (e.g., 1, 2, 3, 4, 5, 6, 7, 8, 9, 10, or more serine residues), and wherein such phosphorylation is capable of promoting ARF-topoisomerase I complex formation. Suitable serine kinases include, for example, casein kinase II (CKII) or protein kinase C (PKC).

In other embodiments, the agent is a vector encoding ARF, or a biologically active fragment thereof, operably linked to a promoter. Preferably, the biologically active ARF fragment contains amino acid residues 66-84 of ARF.

In other embodiments, the cancer is, for example, lung cancer, prostate cancer, hepatocellular carcinoma, breast cancer, colorectal cancer, acute myelogenous leukemia, melanoma, ovarian cancer, neuroendocrine carcinoma, gastric cancer, esophageal cancer, pancreatic cancer, or adenocarcinoma.

In other embodiments, the patient is further administered with at least one other chemotherapeutic agent including, for example, an alkylating agent, an anti-metabolite, a vinca alkaloikd, or an anti-tumor antibody. In other embodiments, the patient is administered anti-cancer radiation therapy prior to, concurrent with, or subsequent to administration of the topoisomerase I inhibitor.

Suitable topoisomerase I inhibitors stabilize the topoisomerase I-DNA complex. Preferable topoisomerase I inhibitors include, for example, camptothecin, irinotecan, topotecan, and analogs thereof.

As used herein, “topoisomerase I” refers to human topoisomerase I found at Gen bank accession no NM_{—}003283 (FIG. 10).

As used herein, “p14ARF (ARF)” refers to the human ARF protein found at Genbank accession no. NP_{—}478102 (FIG. 9) and its homologs. It is believed that ARF interacts with, and activates, topoisomerase I.

Biologically active fragments of ARF contain substantially all of the topoisomerase binding domain (i.e., amino acid residues 66-84) responsible for topoisomerase I binding. In all cases, the ARF polypeptide must be capable of binding to phosphorylated topoisomerase I. Suitable biologically active fragments include, for example, an N-terminal truncation of the ARF protein (e.g., amino acid residues 66-132), or a polypeptide fragment or chimeric protein containing substantially all of the topoisomerase I binding domain (amino acid residues 66-84).

By “serine kinase biological activity” is meant any enzymatic activity that is capable of phosphorylating a serine amino acid residue on a target protein. Typically, this is an ATP-dependent reaction in which the γ-phosphate group of an ATP molecule is transferred to the serine residue of the substrate protein. Preferred serine kinases include, for example, CKII and PKC.

By “increased serine kinase biological activity,” when referring to the serine kinase biological activity within a cell in accordance with the principles of this disclosure, is meant a level of serine kinase biological activity in the cell nucleus which, following a specific treatment or intervention, is higher than would otherwise be present in the same cell absent that specific treatment or intervention (i.e., the basal level). Elevated serine kinase biological activity is preferably at least 10%, 20%, 30%, 40%, 50%, 75%, 100%, 200%, or more greater than the basal serine kinase biological activity level. Elevated serine kinase biological activity is determined using an assay which directly measures phosphorylation events attributable to the kinase activity.

A suitable assay for PKC biological activity is described in U.S. Pat. No. 5,538,858 (hereby incorporated by reference) and is based on the Amersham Protein Kinase Enzyme Assay Kit RPN77. Nuclear extracts are prepared from treated and untreated cells. Suitable methods for preparation of nuclear extracts are described in (Olnes, et al., Biotechniques 1994;17(5):828-9). Briefly, the assay buffer is created by mixing equal amount of calcium buffer (12 mM calcium acetate in 50 mM Tris buffer, pH 7.5), lipid buffer (0.3 mg/ml Lα-phosphatidyl-L-serine and 24 μg/ml phorbol 12-myristate-13-acetate in 50 mM Tris buffer pH 7.5), peptide buffer (900 μM RKRTLRRL (SEQ ID NO.: 1) in 50 mM Tris buffer pH 7.5), and DTT buffer (30 mM dithiothreitol in 50 mM Tris buffer pH 7.5). An equal amount of assay buffer is then added to equal amounts of the nuclear extracts and ATP buffer (1.2 mM ATP, γ-³²P-ATP (sufficient to result in about 0.2 μCi per assay), 30 mM Hepes, 72 mM magnesium acetate, pH 7.5). The assays are incubated for about 15 minutes at about 37° C. The kinase reaction is stopped by the addition of a sufficient volume of ice-cold orthophosphoric acid (300 mM). The resulting products are filtered (trapping the peptide substrate), washed, and the level kinase activity determined by measuring the level of ³²P incorporation into the peptide substrate (e.g., using scintillation counting).

A suitable assay for CKII biological activity is described in conjunction with the Casein Kinase II Assay kit available from Sigma-Aldrich (Product #CS 0610). Again, nuclear extracts are used in the kinase assay. Briefly, 15 μl of Buffer A (200 mM Hepes, 650 mM KCl, 50 mM MgCl₂, 0.05 mM ATP, γ-³²P-ATP (sufficient to result in about 0.2 μCi per assay), 25 mM DTT, 25 mM β-glycerophosphate, 1 mM sodium orthovanadate, pH 7.5) is mixed with 10 μl of 100 mM Hepes (pH 7.5), 15 μl water, and 10 μl (or other suitable amount) of nuclear extract (prepared as described herein). The assays are incubated for about 15 minutes at about 37° C. The kinase reaction is stopped adding trichloroacetic acid and spotting the reaction mixture on a cellulose phosphate filter paper. The filters are washed using 0.5% phosphoric acid and the level kinase activity determined by measuring the level of 32P incorporation into the peptide substrate (e.g., using scintillation counting).

The skilled artisan recognizes that there exist a variety of kinase assays for measuring the activity of PKC, CKII, as well as other serine kinases of interest. Alternatively, serine kinase biological activity can be measured indirectly by measuring elevated levels of one or more phospho-proteins which are known to be phosphorylated by the kinase of interest. For example, as described herein, the levels of phosphorylated topoisomerase I was assessed by immunoprecipitation using an antibody that binds to both the phosphorylated and unphosphorylated form of the protein, followed by Western blotting using a phosphoserine-specific antibody. Western blots are amenable to relative quantification by densitometric analysis.

By “phosphorylates topoisomerase I”, when referring to a serine kinase enzyme, is meant any serine kinase enzyme which is capable of catalyzing a phosphotransferase reaction involving the transfer of the γ-phosphate group of ATP or other nucleoside triphosphate to a serine residue of the topoisomerase I enzyme. The capability of a serine kinase (or any enzyme) to phosphorylate topoisomerase I can be determined using any kinase assay described herein or any other suitable assay known in the art for that particular kinase. A suitable kinase substrate representing the serine amino acid phosporylating site in topoisomerase I is a polypeptide of not less than 10 amino acids, having at least one a serine residue no less than four amino acid residues from either terminus of the polypeptide, and wherein the polypeptide is identical to a portion of the human topoisomerase I enzyme (SEQ ID NO: 3).

By “topoisomerase I inhibitor” is meant a compound that is capable of inhibiting the DNA re-ligation enzymatic reaction catalyzed by topoisomerase I. Preferred topoisomerase I inhibitors are capable of creating a stabilized DNA-topoisomerase I complex sufficient to inhibit the enzymatic reaction. In order to determine whether a compound of interest is a topoisomerase I inhibitor, the relaxing of supercoiled DNA is measured in the presence of topoisomerase I and the compound of interest. The result is compared to an assay performed under the same conditions in the absence of the compound of interest, wherein a topoisomerase I inhibitor reduces or prevents relaxation of the supercoiled DNA. A suitable assay for measuring topoisomerase I inhibition is described in the Examples contained herein. Topoisomerase I inhibitors include, for example, plant alkaloids, plant alkaloid derivatives, camptothecin, irinotecan, topotecan, and analogs thereof.

By “stabilized complex” is meant a DNA-topoisomerase I complex in which the topoisomerase I catalytic activity has been partially or completely inhibited by the further binding of a topoisomerase I inhibitor. Normally, the DNA-topoisomerase I complex is a transient chemical intermediate species formed during the isomerase reaction. But, in the presence of a topoisomerase I inhibitor, isomerization, DNA ligation, and/or DNA release is inhibited, resulting in a stabilized complex which inhibits DNA replication.

By “contacting”, when referring to the interaction between a cell and an agent, is meant a physical interaction between the cell (or a cellular component) and the agent such that the desired biological effect is produced as a direct or indirect result of that interaction. Contacting may involve, for example, a physical interaction between the agent and a cell surface receptor, followed by a signal transduction event in resulting in the desired biological activity within the cell. Alternatively, contacting may require internalization of the agent in order for the biological effect to be produced. Such is the case for vectors encoding serine kinase enzymes or ARF.

By a “vector” is meant a non-chromosomal nucleic acid comprising an intact replicon such that the vector may be replicated when placed within a cell, for example by a process of transformation, transfection or transduction. Vectors may be viral or non-viral. Viral vectors include retroviruses, adenoviruses, herpesvirus, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

By a “promoter” is meant a nucleic acid sequence sufficient to direct transcription of a gene. Also included in the invention are those promoter elements which are sufficient to render promoter dependent gene expression controllable for cell type specific, tissue specific or inducible by external signals or agents (e.g. enhancers or repressors); such elements may be located in the 5′ or 3′ regions of the native gene, or within an intron.

By “operably linked” is meant that a nucleic acid molecule and one or more regulatory sequences (e.g., a promoter) are connected in such a way as to permit expression and/or secretion of the product (e.g., a protein) of the nucleic acid molecule when the appropriate molecules (e.g., transcriptional activator proteins) are bound to the regulatory sequences.

In another aspect, the invention provides a method for inducing apoptosis, cell killing, and/or growth arrest in a cell by contacting the cell with an agent that inhibits the binding of ARF to topoisomerase I. The binding may be inhibited by an antibody or other binding agent (e.g. a peptide, an aptamer, or a peptidomimetic) which disrupts the interaction between ARF to topoisomerase I. The agent may bind directly to ARF or to topoisomerase I and may competitively or non-competitively inhibit the ARF-topoisomerase I binding interaction. Suitable antibodies include, for example, ARF-specific antibodies and topoisomerase I-specific antibodies. Alternatively, a phosphatase that dephosphorylates topoisomerase I may be used to reduce ARF binding to topoisomerase I. In preferred embodiments, the method disrupts existing ARF-topoisomerase I complexes. In other embodiments, ARF binding to HDM2 is increased. In other embodiments, p53 biological activity is increased.

In another aspect, the invention provides methods for determining the sensitivity of a cancer cell to a topoisomerase I inhibitor comprising: (i) determining the nuclear localization of ARF within the cancer cell, and (ii) identifying the cancer cell as being sensitive to a topoisomerase I inhibitor when the ARF is substantially localized to the nucleolus and identifying a cancer cell as being resistant to a topoisomerase I inhibitor when said ARF is substantially disbursed in the nucleus of said cell. In this context, the term “substantially” means greater than 50%. In preferred embodiments in which cancer cells are identified as being sensitive to a topoisomerase inhibitor, more than 50%, 60%, 70%, 80%, 90%, 95%, or 99% of the ARF is present the nucleolus or the nucleolus and perinucleolar region.

In another aspect, the invention provides methods for determining the sensitivity of a cancer cell to a topoisomerase I inhibitor comprising: (i) determining the ratio of free ARF to ARF bound to topoisomerase I in the nucleus of the cancer cell, and (ii) identifying the cancer cell as being sensitive to a topoisomerase I inhibitor when the ratio is less than 1, and identifying a cancer cell as being resistant to a topoisomerase I inhibitor when the ratio is greater than 1. In preferred embodiments in which cancer cells are identified as being sensitive to a topoisomerase inhibitor, the ratio is less than 0.9, 0.8, 0.7, 0.6, 0.5, 0.4, 0.3, 0.2, or 0.1. In preferred embodiments in which cancer cells are identified as being resistant to a topoisomerase inhibitor, the ratio is greater than 2, 3, 4, 5, 7, 10, 20, 25, 50, 90, or 100.

In another aspect, the invention provides methods for determining the sensitivity of a cancer cell to a topoisomerase I inhibitor comprising: (i) determining the ratio of unphosphorylated topoisomerase I to phosphorylated topoisomerase I in the nucleus of the cancer cell, and (ii) identifying the cancer cell as being sensitive to a topoisomerase I inhibitor when the ratio is less than 1, and identifying a cancer cell as being resistant to a topoisomerase I inhibitor when the ratio is greater than 1. In preferred embodiments in which cancer cells are identified as being sensitive to a topoisomerase inhibitor, the ratio is less than 0.9, 0.8, 0.7, 0.6, 0.5, 0.4, 0.3, 0.2, or 0.1. In preferred embodiments in which cancer cells are identifies as being resistant to a topoisomerase inhibitor, the ratio is greater than 2, 3, 4, 5, 7, 10, 20, 25, 50, 90, or 100.

In preferred embodiments of the aspects of this invention, the topoisomerase is camptothecin, irinotecan, and topotecan. In other embodiments, the cancer cell is a lung cancer cell, prostate cancer cell, hepatocellular carcinoma cell, breast cancer cell, colorectal cancer cell, acute myelogenous leukemia cell, melanoma cell, or adenocarcinoma cell, ovarian cancer cell, neuroendocrine carcinoma cell, gastric cancer cell, esophageal cancer cell, or pancreatic cancer

In another aspect, the invention provides kits for determining the sensitivity of a cell (e.g., a cancer cell) to a topoisomerase I inhibitor. The kits comprise (i) an anti-phosphoserine antibody, (ii) an anti-topoisomerase I antibody, and (iii) an anti-ARF antibody. In a preferred embodiment, the anti-topoisomerase I antibody binds to human topoisomerase I. In another preferred embodiment, the anti-ARF antibody binds to human ARF.

In another aspect, the invention provides a cells containing a recombinant vector and a topoisomerase I inhibitor. Suitable recombinant vectors include vectors encoding a serine kinase (e.g., CKII or PKC), ARF, or a biologically active fragment of ARF. In preferred embodiments, the cell further contains a stabilized DNA-topoisomerase I complex.

In another aspect, the invention provides a cell comprising a topoisomerase I inhibitor and further expressing an elevated serine kinase biological activity, wherein the cell has been contacted with an agent that elevates the serine kinase biological activity relative to the serine kinase biological activity in the same cell which has not been contacted with the agent.

### BRIEF DESCRIPTION OF THE DRAWINGS

FIG. 1 is a series of electrophoretic gel separations of nuclear proteins from DU145, H358, and H23 cells. FIG. 1A is a silver-stained electrophoretic gel showing cellular proteins corresponding in size to topoisomerase I that bound to an immobilized fusion protein composed of the N-terminal portion of ARF (ARF-N-term; exon 1β, amino acid residues 1-64) and the full-length ARF protein. FIG. 1B shows a Western analysis of topoisomerase I that bound to immobilized full length ARF or Nickel-NTA agarose lacking immobilized fusion protein (control, middle panel). FIG. 1C shows co-binding of topoisomerase I and ARF following immunoprecipitation of H358 and H23 nuclear extracts with an anti-topo I antibody followed by Western detection of topo I or ARF in the immunoprecipitated material, before (left panel) or 48 hours after treatment with Adp14, moi=20 pfu/cell (middle panel). Far right panel shows material that remained unbound by the anti-topo I antibody.

FIG. 2A is a graph showing that nuclear extracts from H358 cells (closed circles) have greater topoisomerase I activity compared to and H23 cells (open circles) in an in vitro assay measuring the conversion of supercoiled plasmid DNA (“s”) to the relaxed form (“r”). FIG. 2B is an agarose gel electrophoresis of reaction products of a typical in vitro topoisomerase I assay in which 0.32, 0.65, or 1.3 μg of H358 extract (lanes 1-3, respectively) or H23 extract (lanes 4-6, respectively) were added per reaction. FIG. 2C is an agarose gel electrophoresis showing that addition of ARF protein in the topoisomerase activity assay increases the topoisomerase I activity of H358 nuclear extracts (lanes 1-3), but not H23 nuclear extracts (lanes 4-6). The ARF-N-term fusion protein (lanes 7-12) had no effect on topoisomerase I activity.

FIG. 3A is an electrophoretic gel showing that ARF binds to topoisomerase I in H358 nuclear extracts, but not in H23 nuclear extracts. The ARF-topoisomerase I complexes are destroyed by alkaline phosphatase (+AP) treatment and restored in both cell types following CKII treatment. FIG. 3C shows that this effect is also achieved using purified topoisomerase I. FIG. 3C also shows that HT29 cells have low levels of topo I serine phosphorylation and ARF-topoisomerase I complexation relative to H358 cells. This data demonstrates that the ARF-topoisomerase I complex formation is a phosphorylation dependent event. FIG. 3B is an electrophoretic gel showing that the catalytic activity of topoisomerase I in H358 cells is abolished by alkaline phosphatase treatment and the activity cannot be enhanced by overexpression of ARF. FIG. 3D is a bar graph showing the CKII activity in lysates of H358, H23, and HT29 cells.

FIG. 4A is an electrophoretic gel separation and Western of topoisomerase I and ARF following subcellular fractionation. These data show that topoisomerase I is concentrated in the nucleolus of both H538 and H23 cells, and ARF is also concentrated in the nucleolus of H538 cells. By contrast, ARF has is distributed approximately evenly between the nucleolus and the nucleoplasm of H23 cells. FIG. 4B is a series of photomicrographs showing the immunofluorescence pattern of ARF in fixed and permeabilized H358 and H23 cells. This confirms the findings of FIG. 4A and demonstrates that there is reduced nucleolar ARF localization in H23 cells. FIG. 4C is an electrophoretic separation following co-immunoprecipitation analysis of Nucleophosmin (NPM/B23) and ARF in H358 and H23 nuclear extracts.

FIG. 5A is an electrophoretic separation and Western analysis of H358 cellular actin (top row) or ARF (bottom row) 48 hours after treatment with Adp14 (lane 1) or Ad1β(lane 2), or 72 hours after treatment with siRNA control sequence (lane 3), or ARF siRNA to exon 2 (lane 5). Lane 4 shows actin and ARF levels in untreated H358 cells. Digital analyses of ARF band intensities are shown beneath the ARF Western blot. FIG. 5B is a series of graphs showing H358 and PC-3 cell viabilities assayed 5 days post-vector treatment (adenoviral vector, moi 20 pfu/cell, or siRNA), and 4 days post treatment with increasing doses of camptothecin. Viability is expressed as a percent of no-camptothecin control for each vector or siRNA treatment. Results represent average of triplicate wells, with standard deviations indicated. Treatments: Adp14 (ARF full length ▪); Ad1β (ARF N-term ◯); siRNA control (▾); no vector (); siRNA exon 2 (∇). FIG. 5C shows the results of a Western analysis (top panel) of H23 cellular actin and ARF levels in untreated cells (lane 1) or 72 hours after treatment with ARF siRNA to exon 1β (lane 3), or siRNA plus Adp14 (moi=100) (lane 2). Digital analyses of ARF levels are shown below ARF lanes. (lower panel) H358 cell viability assay following the indicated treatments. Viability was measured 3 days post-start of camptothecin treatment. Together, these data demonstrate that reduced ARF levels, and thus reduced ARF-topoisomerase I complex formation (see FIG. 5D), renders cells less sensitive to topoisomerase I inhibitors.

FIG. 5D shows ARF-topoisomerase I complex formation in H358 cells following various treatments, and correlates differences in complex formation with differences in topoisomerase I activity. FIG. 5D (upper panel shows topoisomerase I immunoprecipitation followed by topoisomerase I or ARF Western following various treatments. Lanes correspond to the same treatments as in FIG. 5A. Digital analyses of ARF band intensities are shown below the ARF lanes. FIG. 5D (middle panel) is an ethidium bromide-stained agarose gel of the reaction products of an in vitro topoisomerase I assay measuring loss of supercoiled plasmid DNA in the presence of 0.06 μg H358 nuclear extract (amount that converts 50% of supercoiled plasmid to relaxed form; see FIG. 2A). Numbered lanes correspond to the same treatments as in FIG. 5A. s=supercoiled; r=relaxed form. FIG. 5D (lower panel) is a graphical representation of the relative supercoil band intensities of lanes 1-5 of the ethidium bromide-stained agarose gel shown in the middle panel. These data demonstrate that ARF-topoisomerase I complex formation and topoisomerase I activity were altered in a predictable and coordinate manner by overexpressing or inhibiting ARF. FIG. 5E is a graph showing the H23 cell viability assay performed as described above. Consistent with the observation that topoisomerase I activity in H23 cells is not enhanced by ARF overexpression, this experiment demonstrates that ARF overexpression does not render H23 cells sensitive to topoisomerase I inhibitors.

FIG. 6 is a series of electrophoretic gels showing that ARF binding promotes topoisomerase I complex formation with DNA. The top panel shows the results of an immunodepletion assay carried out on nuclei prepared from cells treated with increasing doses of Adp14, followed by camptothecin treatment to crosslink topoisomerase I onto DNA. The gel shows that increasing levels of ARF lead to a reduction in the band intensity of topoisomerase I, indicating that more topoisomerase I has become covalently bound to DNA by camptothecin and therefore cannot enter the gel. The middle and bottom panels show topoisomerase I immunoprecipitation followed by an ARF and a topoisomerase I Western analysis, respectively, in cells treated with increasing doses of Adp14. Digital analyses of topoisomerase I and ARF levels are shown below lanes. The results show that increasing doses of Adp14 promote increasing levels of ARF-topoisomerase I complex formation, and that this promotes increased topoisomerase I binding to DNA following camptothecin treatment.

FIG. 7 is a series of graphs showing the correlation of serine phophorylation, ARF/topoisomerase I complex formation and camptothecin sensitivity. FIGS. 7A-C show the relative amounts of (A) serine phosphorylation of topoisomerase I, (B) total topoisomerase I, and (C) ARF-topoisomerase I complex following topoisomerase I immunoprecipitation in the indicated cell types. FIG. 7D is a graph showing the viability of the indicated cell types 3 days after treatment with camptothecin.

FIG. 8A is a graph showing the viability of the indicated cell types 3 days after treatment with camptothecin. FIG. 8B is an electrophoretic gel of a topoisomerase I immunoprecipitation followed by an ARF Western analysis. FIG. 8C is an electrophoretic gel of a topoisomerase I immunoprecipiation and a phosphoserine Western analysis. FIG. 8D is an ARF Western analysis. Together, these data demonstrate that the level of ARF-topoisomerase I complex formation in Hela cells is intermediate to that of H538 and H23 cells. The sensitivity of Hela cells to topoisomerase I inhibitors is also intermediate to that of H538 and H23 cells. The fact that Hela cell topoisomerase I is serine phosphorylated indicates that abnormalities other than serine phosphorylation can disrupt the ARF/topoisomerase I complex and promote resistance to camptothecin.

FIG. 9 is the amino acid sequence of human ARF, as provided in accession no. NP_{—}478102 (SEQ ID NO: 2).

FIG. 10 is the amino acid sequence of human topoisomerase I, as provided in accession no NM_{—}003286. (SEQ ID NO: 3).

### DETAILED DESCRIPTION OF INVENTION

The present inventions are based on different mechanisms for inducing apoptosis and/or growth arrest in cancer cells. Each mechanism is based upon altering (i.e., increasing or decreasing) the amount of ARF-topoisomerase I complex formation. One mechanism is based on the discovery that reduced topoisomerase I serine phosphorylation and/or ARF-topoisomerase I complex formation renders cells less sensitive (or insensitive) to the apoptotic and/or growth arresting effects of topoisomerase I inhibitors. Sensitivity to topoisomerase I inhibitors may be restored by increasing amount of ARF-topoisomerase I complex formation which may be done by increasing the serine phosphorylation of the enzyme (e.g., using CKII or PKC), or by increasing ARF in order to promote complex formation. Another mechanism is based on the discovery that disruption of ARF-topoisomerase I complex formation correlates with apoptosis and/or growth arrest.

As described in more detail in the following examples, analysis of the H23 non-small cell cancer cell line identified cancer-related defects in topoisomerase I-ARF binding. Specifically, the loss of topoisomerase I serine phosphorylation caused a corresponding loss of topoisomerase I activity. Additionally, the absence of topoisomerase I serine phosphorylation resulted in reduced ARF binding and caused an aberrant nuclear distribution of ARF. It was further observed in H23 cells that only about half of the cellular ARF was bound to NPM, a nucleolar protein. Normally, virtually all cellular ARF is NPM-bound.

Increased ARF-topoisomerase I Complex Formation Increases Sensitivity to Topoisomerase I Inhibitors.

The mechanisms that regulate topoisomerase I activity are of considerable therapeutic interest, since topoisomerase I has proven to be an important target for chemotherapy (Pommier, et al., Biochim Biophys Acta 1998;1400(1-3):83-105; Liu, L. F., Annu Rev Biochem 1989;58:351-75). A potent class of chemotherapeutic drugs that target topoisomerase I are derived from the plant alkaloid, camptothecin, a group that includes irinotecan (Camptosar) and Topotecan. These agents have been highly effective for the treatment of a variety of solid tumors that have shown resistance to other treatments, including non-small cell lung cancer (Rothenberg, M. L., Oncologist 2001;6(1):66-80). Camptothecin and its derivatives prevent the re-ligation of the cleavable complex, a topoisomerase I reaction intermediate, thereby creating lethal topoisomerase I-induced DNA strand breaks (Champoux, J. J., Annu Rev Biochem 2001;70:369-413). As with many chemotherapeutic treatments, however, de novo or acquired resistance to camptothecins is common, and can occur through a variety of mechanisms (Rasheed, et al., Oncogene 2003;22(47):7296-304; Xu, et al., Ann Oncol 2002;13(12):1841-51), including downregulation of topoisomerase I activity (Pommier, et al., Ann N Y Acad Sci 1996;803:60-73).

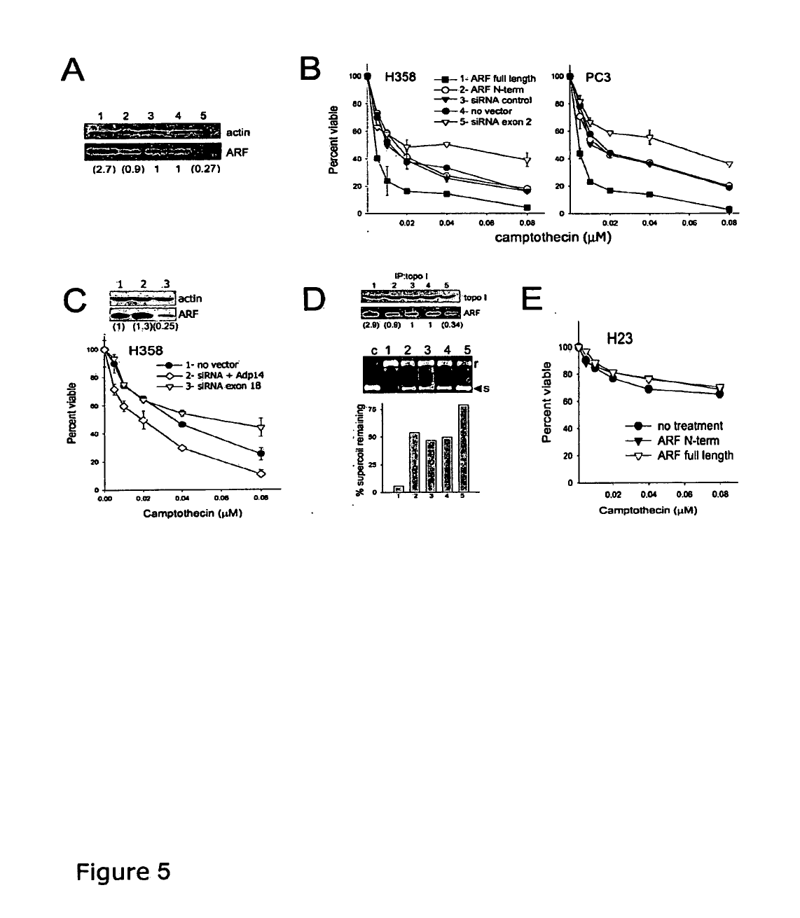

The following examples demonstrate that reduced levels of topoisomerase I activity and failure of ARF/topoisomerase I complex formation in H23 cells correlates with camptothecin resistance, while ectopic over expression of ARF and increased ARF/topoisomerase I complex formation in H358 cells results in enhanced camptothecin sensitivity (FIG. 5).

Without wishing to be bound by any theory, it is believed that the apoptosis, cell killing and/or growth arrest caused by topoisomerase I inhibitors requires a catalytically active topoisomerase I enzyme. Catalytic activity is enhanced by ARF-topoisomerase I complex formation, which itself requires serine phosphorylation of the enzyme. Thus, the following examples demonstrate that ARF-topoisomerase I complex formation can be increased by increasing the amount of serine phosphorylation of the enzyme and/or increasing the amount of ARF (or a biologically active fragment of ARF) available for topoisomerase I binding. The resulting elevation in ARF-topoisomerase I complex formation increases the sensitivity of the cell to topoisomerase I inhibitors which bind to, and stabilize, the covalent complex formed as an intermediate during the isomerase reaction. The stabilized complexes likely prevent further DNA replication.

Disruption of ARF-topoisomerase I Complex Formation Induces Apoptosis and/or Growth Arrest in Cancer Cells.

ARF is a well known positive regulator of the p53 tumor suppressor. ARF interacts with and sequesters human double minute (HDM2) or its equivalent, a negative regulator of p53. In doing so, ARF promotes the accumulation of p53 protein which results in p53-mediated cell cycle arrest or apoptosis.

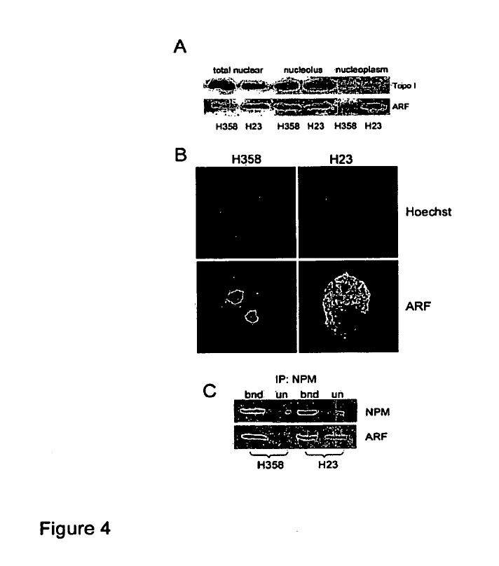

As demonstrated herein, ARF is normally localized to the nucleolus as a result of its topoisomerase I binding. This effectively prevents ARF from binding to HDM2, thereby permitting HDM2-inhibition of p53. However, disruption of the ARF-topoisomerase I binding interaction allows ARF to redistribute from the nucleolus to the nucleoplasm (FIG. 4). Without wishing to be bound by any theory, it is believed that this redistribution allows ARF to bind and sequester HDM2, causing a dis-inhibition of p53. It is this p53 activation which underlies the apoptotic and growth arresting effect caused by the disruption of ARF-topoisomerase I complex formation.

Vectors Suitable for Delivery to Humans

This invention features methods and compositions for treating cancer. The cancer may be treated by inducing cell death (e.g., apoptosis) or growth arrest in the cancer cells. In one aspect, the invention features methods of gene therapy to express ARF or a serine kinase (e.g., CKII or PKC) in the cancer cells of a patient. Gene therapy, including the use of viral vectors as described herein, seeks to transfer new genetic material (e.g., polynucleotides encoding a serine kinase) to the cells of a patient with resulting therapeutic benefit to the patient. For in vivo gene therapy, expression vectors encoding the gene of interest is administered directly to the patient. The vectors are taken up by the target cells and the serine kinase gene expressed. Several recent reviews are available discussing methods and compositions for use in gene therapy (Eck et al., in Goodman & Gilman's The Pharmacological Basis of Therapeutics, Ninth Edition, Hardman et al., eds., McGray-Hill, New York, 1996, Chapter 5, pp. 77-101; Wilson, Clin. Exp. Immunol. 107 (Suppl. 1):31-32, 1997; Wivel et al., Hematology/Oncology Clinics of North America, Gene Therapy, S. L. Eck, ed., 12(3):483-501, 1998; Romano et al., Stem Cells, 18:19-39, 2000, U.S. Pat. No. 6,080,728).

Adenoviruses are able to transfect a wide variety of cell types, including non-dividing cells. There are more than 50 serotypes of adenoviruses that are known in the art, but the most commonly used serotypes for gene therapy are type 2 and type 5. Typically, these viruses are replication-defective; genetically modified to prevent unintended spread of the virus. This is normally achieved through the deletion of the E1 region, deletion of the E1 region along with deletion of either the E2 or E4 region, or deletion of the entire adenovirus genome except the cis-acting inverted terminal repeats and a packaging signal (Gardlik et al., Med. Sci. Monit. 11: RA110-121, 2005).

Retroviruses are also useful as gene therapy vectors and usually (with the exception of lentiviruses) are not capable of transfecting non-dividing cells. The invention includes use of any appropriate type of retrovirus that is known in the art, including, but not limited to, HIV, SIV, FIV, EIAV, and Moloney Murine Leukaemia Virus (MoMLV). Typically, therapeutically useful retroviruses including deletions of the gag, pol, or env genes.

Adeno-associated virus (AAV) vectors can achieve latent infection of a broad range of cell types, exhibiting the desired characteristic of persistent expression of a therapeutic gene in a patient. The invention includes the use of any appropriate type of adeno-associated virus known in the art including, but not limited to AAV1, AAV2, AAV3, AAV4, AAV5, and AAV6 (Lee et al., Biochem J. 387: 1-15, 2005; U.S. Patent Publication 2006/0204519).

Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA. In vivo DNA-mediated gene transfer into a variety of different target sites has been studied extensively. Naked DNA may be administered using an injection, a gene gun, or electroporation. Naked DNA can provide long-term expression in muscle (Wolff, et al., Human Mol. Genet., 1:363-369, 1992; Wolff, et al., Science, 247, 1465-1468, 1990). DNA-mediated gene transfer has also been characterized in liver, heart, lung, brain and endothelial cells (Zhu, et al., Science, 261: 209-211, 1993; Nabel, et al., Science, 244:1342-1344, 1989). DNA for gene transfer also may be used in association with various cationic lipids, polycations and other conjugating substances (Przybylska et al., J. Gene Med., 6: 85-92, 2004; Svahn, et al., J. Gene Med., 6: S36-S44, 2004).

Methods of gene therapy using cationic liposomes are also well known in the art. Exemplary cationic liposomes for use in this invention are DOTMA, DOPE, DOSPA, DOTAP, DC-Chol, Lipid GL-67.TM., and EDMPC. These liposomes may be used to encapsulate a serine kinase vector for delivery into target cells.

Typically, vectors made in accordance with the principles of this disclosure will contain promoters that will cause constitutive expression of the serine kinase coding sequence, although inducible promoters may be used.

Administration of Topoisomerase I inhibitors

In addition to elevating the serine kinase levels (e.g., CKII and PKC levels) in a cancer cell, sufficient to increase phosphorylation of topoisomerase I, or increasing ARF levels sufficient to enhance the formation of an ARF/topoisoermase I complex it is desirable that the cancer cells be further contacted with one or more topoisomerase I inhibitors. Typically, patients diagnosed as having cancer will be administered a pharmaceutical formulation containing a topoisomerase I inhibitor. Suitable topoisomerase I inhibitors include, for example, camptothecin, irinotecan, topotecan, and analogs of these inhibitors. The administration of topoisomerase I inhibitors may be by any suitable means that results in an anti-neoplastic effect. The topoisomerase I inhibitor may be administered in any appropriate amount, in any suitable carrier substance, and is generally present in an amount of 1-95% by weight of the total weight of the composition. The composition may be provided in a dosage form that is suitable for the oral, parenteral (e.g., intravenously, intramuscularly), rectal, or transdermal administration. Thus, the composition may be in form of, e.g., tablets, capsules, pills, powders, granulates, suspensions, emulsions, solutions, gels including hydrogels, pastes, ointments, creams, suppositories, enemas, or injectables. The pharmaceutical compositions may be formulated according to conventional pharmaceutical practice (see, e.g., Remington: The Science and Practice of Pharmacy, (19th ed.) ed. A. R. Gennaro, 1995, Mack Publishing Company, Easton, Pa. and Encyclopedia of Pharmaceutical Technology, eds. J. Swarbrick and J. C. Boylan, 1988 1999, Marcel Dekker, New York.

Camptothecin, irinotecan, topotecan and their analogs, for example, may be administered at doses of about 0.1-1000 mg/kg/day (e.g., about 1, 10, 25, 50, 75, 100, 250, 500, 750, or 1000 mg/kg/day) (U.S. Pat. Nos. 5,004,758, 5,340,817, 5,633,016, 5,859,022, 5,910,491, 6,040,306, 6,214,821, 6,534,080; hereby incorporated by reference). Administration of any of the topoisomerase I inhibitors described herein may continue for about a week, a month, six months, a year, or even the lifetime of the patient.

### EXAMPLES

### Example 1

### Defective ARF/topoisomerase I Complex Formation in H23 Cells.

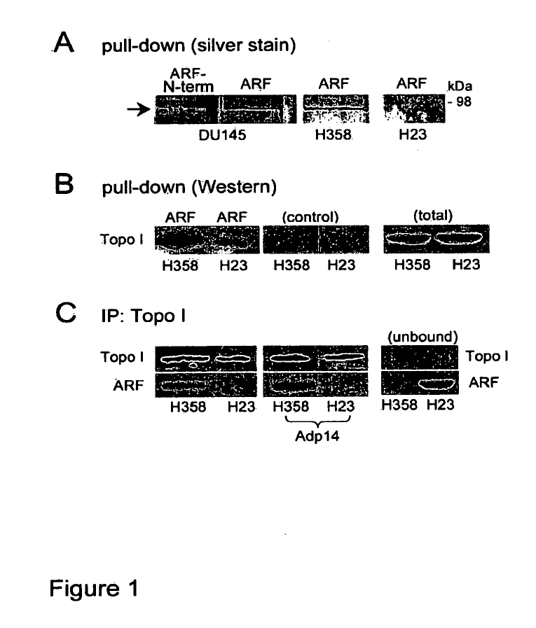

FIG. 1A shows a silver stained gel following a pull-down assay in which immobilized human ARF-thioredoxin fusion protein (or the N-terminal domain (1-64) of ARF) was used to compare ARF-binding proteins from DU145 (prostate cancer), H358, and H23 (non-small cell lung carcinoma)cell RIPA lysates.

Topoisomerase I bound to full-length ARF (ARF, FIG. 1A) but not the ARF N-terminal domain (ARF-N-term, amino acid residues 1-64, FIG. 1A) encoded by ARF's first exon (exon 1β). This is consistent with previous reports that topoisomerase I binds to ARF through the ARF C-terminal, exon 2-encoded domain (Ayrault, et al., Oncogene 2006;25(19):2827 (correction); Olivier, et al., Oncogene 2003;22(13):1945-54). H23 cells appeared to have significantly less topoisomerase I activity compared to that measured in H358 cells (FIG. 1A, far right lane).

Western blot analysis confirmed that the level of topoisomerase I was reduced in the fraction pulled down by immobilized ARF from H23 cells compared to H358 cells (FIG. 1B, left panel). However, total endogenous topoisomerase protein levels in H23 and H358 cells RIPA lysates were similar (FIG. 1B, right panel). Furthermore, a complete sequence analysis of the 2,295 base pair coding sequence of topoisomerase I in H23 cells showed that the sequence corresponded to the wild-type topoisomerase I sequence (EC.5.99.1.2, Accession # NM_{—}003286). Thus, reduced binding of topoisomerase I from H23 cells to immobilized ARF is not the result of reduced cellular levels of topoisomerase I nor is it the result of a mutation in topoisomerase I that could alter its binding properties.

FIG. 1C shows the results of a co-immunoprecipitation experiment using DNAse I solubilized nuclear extracts. This cellular fraction contains more than 95% of topoisomerase I and ARF (Ayrault, et al., Oncogene 2004;23(49):8097-104). ARF-topoisomerase I complexes were readily detectable in H358 nuclear extracts, but were undetectable in H23 nuclear extracts (left panel, FIG. 1C). Thus, the failure of topoisomerase I from H23 cell lysates to bind immobilized ARF is reflected in the lack of endogenous ARF/topoisomerase I complexes.

To determine whether overexpressed ectopic ARF could drive topoisomerase I into complexes with ARF in H23 and H358 cells, cells were treated with an Adp14 adenoviral vector (moi, 20 pfu/cell) for 4 hours and prepared nuclear extracts 48 hours later. Co-immunoprecipitation analysis followed by Western analysis showed that ARF-topoisomerase I complexes increase about 3 fold in H358 cells following treatment with Adp14; indicating that not all cellular topoisomerase I had been bound by ARF in untreated cells (FIG. 1C middle panel). ARF-topoisomersase I complexes remained undetectable in H23 cells (FIG. 1C middle panel).

The material that remained unbound following two successive immunoprecipitation with anti-topoisomerase I was also analyzed (FIG. 1C, right-hand panel (unbound)). Undetectable amounts of ARF protein in H358 cells were found in the unbound material, indicating that virtually all cellular ARF was complexed with topoisomerase I. In contrast, in H23 cells, virtually all the cellular ARF was found in the unbound material. Taken together, these result demonstrate that the failure of H23 cells to form ARF-topoisomerase I complexes, which are required for topoisomerase I activity, are not a result of reduced ARF or topoisomerase levels, nor are they a result of inactivating mutations in either protein.

Vectors: The Adp14 vector encoding full-length ARF, the Ad1β vector encoding the 64-amino acid residue N-terminal domain of ARF (ARF N-term), and vector treatment conditions have been described (Saadatmandi, et al., Cancer Gene Ther 2002;9(10):830-9; Huang, et al., Cancer Research 2003;63:3646-3653). Equal titers of Adp14 and Ad1β were confirmed by RT-PCR to produce equivalent levels of ARF and ARF N-term message. An siRNA expression plasmid specific for the exon 2-encoded region of ARF (pKD-Ink4a-v2), as well as a negative control siRNA expression plasmid (pKD-NegCon-v1) were purchased from Upstate, Temecula, Calif., and transfected into cells using Lipofectamine™ (Invitrogen, Carlsbad, Calif.) according to the manufacturer's instructions. An siRNA to the exon 1β region of ARF (sense sequence: 5′-GGGUUUUCGUGGUUCACAUtt-3′ (SEQ ID NO: 4); antisense sequence: 5′-AUGUGAACCACGAAAACCCtc-3′ (SEQ ID NO: 5)) was purchased from Ambion, Inc., Austin Tex.

Co-Immunoprecipitation/Western: DNAse I-solubilized nuclear extracts were prepared according to reference (Ayrault, et al., Oncogene 2004;23(49):8097-104). Briefly, cells (10⁶) were harvested and lysed in DNAse I solubilization buffer (10 mM Hepes pH 7.5, 100 mM NaCl, 300 mM sucrose, 3 mM MgCl₂, 1 mM EDTA, 1 mM DTT, 0.5% Triton X-100, 1 mM phenylmethylsulfonyl fluoride (PMSF), complete protease inhibitors (Roche, Nutley, N.J.)), followed by centrifugation to pellet nuclei. Nuclei were resuspended in 300 μL of the same buffer, treated with 1 mg/mL DNAse I (Sigma, St. Louis, Mo.) for 15 minutes at 37° C., and centrifuged. The DNAse I-solubilized material, which contained the bulk of cellular topoisomerase I and ARF protein (Ayrault, et al., Oncogene 2004;23(49):8097-104), was used for immunoprecipitation. (We found that high salt-extracted nuclei (see subnuclear fractionation and topoisomerase I assays, below) and DNAse I-solubilized nuclei were similar with respect to topoisomerase I and ARF recovery; however, DNAse I solubilization avoided the use of high salt concentrations that would disrupt complexes). Co-immunoprecipitation was carried out in 1 mL of the same buffer, overnight at 4° C. with rocking, containing 175 μg protein and 20 μL of antibody following our previously described procedure (Lee, et al., Cancer Res 2005;65(21):9834-42). Where bound and unbound fractions were to be compared, the extracts were subjected to 2 successive treatments with antibody (anti-topoisomerase I or anti-NPM), were found to be sufficient to deplete extracts of immunoreactive material. The immunoprecipitated material from the 1^{st} and 2^{nd} treatments was pooled and designated “bound”. Immunoprecipitates were incubated an additional hour in the presence of 80 μL protein G agarose (Santa Cruz Biotechnology), centrifuged and washed with PBS, resuspended in SDS-PAGE sample buffer, boiled, electrophoresed on a 12.5% SDS-PAGE gel, and subjected to Western analysis as described previously (Saadatmandi, et al., Cancer Gene Ther 2002;9(10):830-9). The material that did not immunoprecipitate was designated “unbound” and was concentrated by precipitation with 5 volumes of acetone, prior to resuspension in sample buffer. Antibodies were: Goat polyclonal anti-topoisomerase I (Santa Cruz Biotechnology, Santa Cruz, Calif.), mouse monoclonal anti-nucleophosmin (NPM, B23) (Sigma (St. Louis, Mo.)), rabbit polyclonal anti-full length ARF (Zymed Laboratories, Inc, South San Francisco, Calif.), mouse monoclonal anti-phosphoserine (Sigma, St. Louis, Mo.). All primary antibodies were used at 1:100 for Westerns. Secondary antibodies for Westerns were goat anti-rabbit, goat anti-mouse, and donkey anti-goat (all purchased from Santa Cruz Biotechnology, Santa Cruz, Calif.) and were used at 1:1000.

Subnuclear fractionation: Isolation of nuclei and preparation of nuclear extracts were carried out as described in reference (Olnes, et al., Biotechniques 1994;17(5):828-9), by swelling cells in hypotonic buffer (10 mM Hepes pH 7.9, 10 mM KCl, 0.1 mM EDTA, 0.1 mM EGTA, 1 mM DTT, 0.5 mM Phenylmethylsulfonyl fluoride (PMSF), complete protease inhibitors (Roche, Nutley, N.J.)), and lysing cells by adding 0.6% NP40 to the hypotonic buffer, followed by centrifugation to recover nuclei. For the topoisomerase I assays, nuclei were then extracted for 1 hour on ice in high salt buffer (20 mM Hepes pH 7.9, 0.4 M NaCl, 1 mM EDTA, 1 mM EGTA, 1 mM DTT, 1 mM PMSF, 10% glycerol, and complete protease inhibitors). For storage, nuclear extracts were adjusted to 50% glycerol and placed at −80° C. until used. For preparation of nucleolar and nucleoplasmic fractions, the NP40-prepared nuclei were centrifuged through sucrose, sonicated, and fractionated by centrifugation again through sucrose as described in reference (Andersen, et al., Curr Biol 2002;12(1):1-11). Nucleoli were recovered in the pellet, and the unpelleted material (nucleoplasm) was concentrated by precipitation with 5 volumes of acetone.

### Example 2

### H23 Nuclear Extracts Have Reduced Topoisomerase I Activity Which Cannot be Stimulated by ARF.

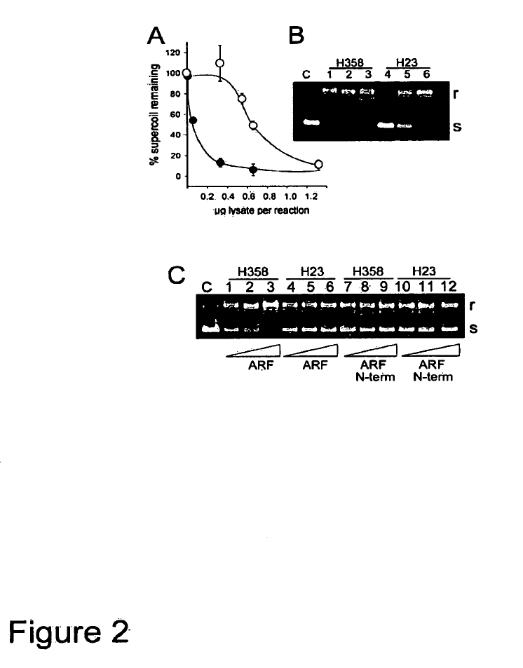

H23 and H358 nuclear extracts were compared for topoisomerase I activity in vitro, and investigated whether the activities could be stimulated by the addition of recombinant thioredoxin-ARF. As shown in FIG. 2A, H358 topoisomerase I was found to be more effective at relaxing supercoiled plasmid DNA than was H23 topoisomerase, achieving 50% relaxation at about 0.06 μg nuclear extract per reaction, some 10-fold lower than the amount of H23 extract needed to achieve the same level of relaxation (0.6 μg extract per reaction). A typical electrophoretic profile of the reaction products with increasing amounts of nuclear extract is shown in FIG. 2B in which 0.32, 0.65, or 1.3 μg of H358 cell extract (lanes 1-3) or H23 (lanes 4-6) were added in each reaction. “r” is the relaxed (non-supercoiled) plasmid and “s” is the supercoiled form.

Similar assays were carried out using the amount of each respective nuclear extract that produced a 50% conversion of supercoiled to relaxed form (0.06 and 0.6 μg extract protein per reaction for H358 and H23, respectively), and added increasing amounts of purified thioredoxin-ARF (3, 9, 27 ng). As a control, in separate assays, increasing amounts of thioredoxin-ARF-N-terminus, which does not bind to topoisomerase I, was added. Thioredoxin-ARF enhanced the activity of H358 topoisomerase in a dose-dependent manner (FIG. 2C, lanes 1-3), but had no effect on H23 topoisomerase (FIG. 2C, lanes 4-6), as expected based on the inability of ARF to bind to H23 topoisomerase. Neither H358 not H23 topoisomerase activities responded to the addition of thioredoxin ARF-N-terminus at similar doses (FIG. 2C lanes 7-12).

Topoisomerase I assays: Assays were carried out using the Topoisomerase I Assay Kit (Topogen, Fla.), according to the instructions provided with the kit and using the high salt nuclear extracts prepared as described above. Briefly, 0.125 μg supercoiled plasmid DNA was incubated with 0-1.3 μg of nuclear extract for 30 minutes at 37° C. The reaction was stopped by adding stop loading dye supplied in the kit and electrophoresed on a 1% agarose/TAE (10 mM TRIS-acetate/1 mM EDTA) gel until the dye front had reached the bottom of the gel. The gel was then stained for 20 minutes in 0.5 μg/mL ethidium bromide, destained in deionized water for 30 minutes, and electrophoresed for an additional hour to enhance band visibility. The gel was photographed and band intensities were analyzed digitally using a Kodak digital camera and analysis software. Some assays were carried out with alkaline phosphatase-treated extracts, prepared as described below, or in the presence of ARF or ARF1β thioredoxin fusion proteins (3, 9, 27 ng), prepared as described above (see pull-down assays).

### Example 3

### Topoisomerase I Activation Requires both Phosphorylation and ARF Binding

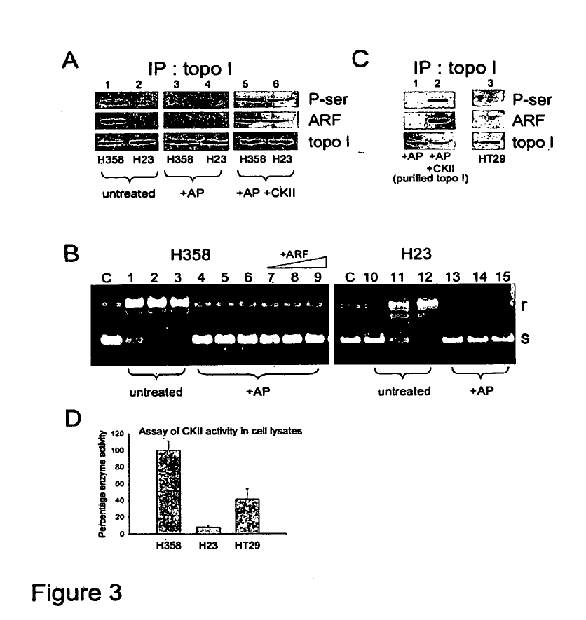

A topoisomerase I immunoprecipitation analysis followed by Western detection of phosphoserine revealed that H358 cells expressed a serine-phosphorylated topoisomerase I (FIG. 3A, lane 1, top row). A similar analysis of phosphotyrosine revealed no evidence for tyrosine phosphorylation (data not shown). Similar results were found in PC-3 cells (data not shown). In contrast, serine-phosphorylated topoisomerase I was only weakly detectable in H23 cells (FIG. 3A, lane 2, top row).

Treatment of both H358 and H23 nuclear extracts with alkaline phosphatase (AP) eliminated serine phosphorylation (FIG. 3A, lanes 3, 4, top row) and abolished their topoisomerase I activity in vitro (FIG. 3B, lanes 4-6 and lanes 13-15). The dephosphorylated topoisomerase I from H358 cells could no longer be activated by addition of increasing amounts of ARF fusion protein (FIG. 3B, lanes 7-9). Furthermore, while topoisomerase I co-immunoprecipitated with ARF from untreated H358 nuclear extracts (FIG. 3A, lane 1, middle row), it failed to co-immunoprecipitate with ARF from H358 nuclear extracts treated with alkaline phosphatase (FIG. 3A, lane 3, middle row). Topoisomerase I failed to co-immunoprecipitate with ARF from either untreated or alkaline phosphatase-treated H23 cell nuclear extracts (FIG. 3A, lanes 2,4, middle row).

When alkaline phosphatase-treated extracts from either H358 or H23 cells were treated with casein kinase II (CKII), a serine phosphorylase, we observed restoration of serine phosphorylation (FIG. 3A, lanes 5,6, top row) and restoration of ARF/topoisomerase I complex formation (FIG. 3A, lanes 5,6, middle row). Recovery of topoisomerase I following immunoprecipitation was the same in all cases (FIG. 3A, lanes 1-6, bottom row).

Alkaline phosphatase treatment of purified recombinant human topoisomerase I, abolished serine phosphorylation (FIG. 3C, lane 1, top row), and abolished its ability to bind recombinant ARF fusion protein (FIG. 3C, lane 1, middle row). But, serine phosphorylation and ARF binding could be restored by treatment with casein kinase II (FIG. 3C, lane 2, top and middle rows, respectively). Recovery of topoisomerase I following immunoprecipitation was the same in all cases (FIG. 3C, lanes 1,2 bottom row). Finally, a topoisomerase I IP/Western analysis was carried out on lysates of an additional cell line, HT29, of colon adenocarcinoma origin. The results revealed a reduced level of serine phosphorylated topoisomerase I that correlated with failure to bind ARF, a result similar to what was seen with H23 cells. Taken together, the results establish that differences in topoisomerase I serine phosphorylation account for the differences in ARF/topoisomerase I complex formation in observed in the cell lines examined.

Casein kinase II assays: 10⁶ cells were harvested, resuspended in 400 μl 10 mM Tris pH 7.4, and subjected to 3 cycles of freeze/thaw. 50 μg of cell extract was assayed for casein kinase II (CKII) activity using the CKII Assay kit from Upstate (Temecula, Calif.), following procedures supplied with the kit.

### Example 4

### Variable CKII Levels Account for the Differences in Topoisomerase Activity among Cell Lines.

Additional assays were performed to determine whether the reduced levels of topoisomerase I serine phosphorylation in H23 and HT29 cells could be due to a reduced cellular levels of casein kinase II (CKII). As shown in FIG. 3D, H23 cell lysates display some 7% of the CKII activity of H358, and HT29 cells display some 41% of the activity of H358. The results indicate that low levels of CKII activity are likely to be responsible for the reduced levels of topoisomerase I serine phosphorylation and reduced ARF/topoisomerase I complex formation in H23 and HT 29 cells.

### Example 5

### Phosphorylated Topoisomerase I Retains ARF in the Nucleolus.

Western analyses of ARF in subnuclear fractions, as well as immunofluorescence staining of ARF in fixed H358 and H23 cells was performed to assess whether the interaction between ARF and topoisomerase I affects subnuclear distribution. For Western analyses, nuclei were prepared as for the topoisomerase I assay, followed either by salt extraction to obtain total nuclear proteins, or by further subfractionation into nucleoplasmic and nucleolar fractions.

FIG. 4A shows the results of Western analyses carried out on total nuclear and subnuclear fractions. Topoisomerase I and ARF levels were comparable in H358 and H23 cells (FIG. 4A, left lanes). Cytoplasmic levels of ARF and topoisomerase I were low to undetectable (not shown). Topoisomerase I was concentrated in the nucleolar fraction in both H358 and H23 cells (FIG. 4A, top row). While ARF was also concentrated in the nucleolar fraction in H358 cells, it appeared to be evenly distributed between nucleolar and nucleoplasmic fractions in H23 cells (FIG. 4A, bottom row). This result was confirmed by immunofluorescence microscopy of fixed cells (FIG. 4B).

Nuclei were stained with the DNA stain, Hoechst 33342, which is excluded from nucleolar regions (top panels). Using an anti-ARF antibody, ARF was detected in a predominantly nucleolar staining pattern in H358 cells (FIG. 4B, bottom left). By contrast, in H23 cells, anti-ARF staining was found across the entire nuclear and perinuclear region (FIG. 4B, bottom right). Thus, failure of ARF to bind topoisomerase I correlates with delocalization of ARF throughout the nucleus, suggesting that topoisomerase contributes to the tethering of ARF in the nucleolus.

The interaction between ARF and nucleophosmin (NPM, B23), an abundant nucleolar protein, was examined in H358 and H23 cells. Nuclear extracts of H358 and H23 cells were immunoprecipitated with two successive treatments with anti-NPM antibody, followed by Western detection of NPM and ARF in the pooled immunoprecipitated material (bnd) or in the material that remained unbound following two successive immunoprecipitations (un).

In H358 cells, virtually all of the cellular ARF was recovered in the material that co-immunoprecipitated with NPM, with undetectable levels recovered in the unbound material (FIG. 4C). This result is consistent with a previous report in murine fibroblasts that the majority of cellular ARF is bound to NPM (Bertwistle, et al., Mol Cell Biol 2004;24(3):985-96). In H23 cells, however, ARF was detected in approximately equivalent levels in the NPM-bound and unbound fractions (FIG. 4C), consistent with its decreased nucleolar localization. Because the majority of cellular ARF in H358 cells could also be recovered in complexes with topoisomerase I (FIG. 1C, compare left and right panels), it is possible that topoisomerase I, ARF, and NPM are present together in a larger complex in H358 cells, and that defective binding of ARF to topoisomerase I in H23 cells destabilizes other interactions of ARF within the complex, including the interaction with NPM. Taken together, the results indicate that binding of ARF to topoisomerase I is required to maintain ARF's full nucleolar localization and its interaction with NPM.

### Example 6

### ARF Mediates Sensitivity to Topoisomerase I Inhibitors.

Adenoviral vectors were used to achieve ectopic overexpression of full-length ARF (Adp14) or ARF-N-terminal domain (Ad1β), and RNA interference to down-regulate endogenous expression of ARF. As shown by the Western analysis of H358 cells in FIG. 5A, ARF levels increased by some 3-fold, as determined by digital analysis of band intensities, by 48 hours post-treatment with Adp14 (moi, 20 pfu/cell, FIG. 5A, lane 1), relative to Ad1β-treated cells (FIG. 5A, lane 2) or untreated cells (FIG. 5A, lane 4). By 72 hours post-transfection of an siRNA expression plasmid to ARF exon 2 (FIG. 5A, lane 5), endogenous ARF levels fell to 0.27 that found in untreated cells (FIG. 5A, lane 4) or control siRNA-treated cells (FIG. 5A, lane 3).

Viability assays were performed 24 hours post-vector treatment by exposing cells for 24 hours to increasing doses of camptothecin (a topoisomerase I inhibitor) in triplicate in a 96-well viability assay, and assaying them for viability 5 days post-start of vector treatment (FIG. 5B). For each growth curve, cell viabilities were normalized to the viability of cells treated with vector only (no camptothecin), to enable a direct visualization of the sensitization effect. As shown for H358 cells in FIG. 5B (left assay), treatment of cells with Adp14 resulted in a greater decrease in cell viability with increasing camptothecin concentrations than did camptothecin alone, while treatment of cells with siRNA to reduce ARF expression resulted in a smaller decrease in cell viability with increasing camptothecin concentrations. Ad1β-treated cells overexpressing the ARF-N-terminal domain that does not interact with topoisomerase I, and control siRNA-treated cells in which levels of endogenous ARF remained unaltered, displayed camptothecin responses similar to cells receiving no vector treatment (FIG. 5B, left).

To verify the generality of these observations, the same series of assays were carried out with the PC-3 prostate cancer cell line (FIG. 5B, right), with similar results. PC-3 cells express active, serine phosphorylated topoisomerase I (data not shown). The siRNA used to down-regulate endogenous ARF, targets the exon 2-encoded region of ARF that is shared by the p16INK4A tumor suppressor. While H358 cells express endogenous p16INK4A, PC-3 cells do not (Chi, et al., Clin Cancer Res 1997;3(10):1889-97), and they therefore provide a control showing that the observed effect on camptothecin sensitivity can be attributed to ARF, and is not cell specific. As a further siRNA control, we reduced endogenous ARF expression in H358 cells by treating them with an siRNA to exon 1β, which is not shared with p16INK4A, and then restored ARF expression by treatment with Adp14 one day later. As shown in the Western analysis in FIG. 5C, siRNA treatment (lane 3, ARF) reduced ARF protein levels to about 0.25 that of untreated cells (lane 1, ARF) by 72 hours post-siRNA treatment. Digital analyses of ARF band intensities are shown below the ARF lanes. Treatment with Adp14 (moi, 100 pfu/cell) 24 hours after siRNA treatment, restored ARF expression, measured 72 hours post-siRNA treatment, to 1.3-fold that found in untreated cells (lane 2, ARF). Actin levels remained unchanged by these treatments (FIG. 5C, actin).

To assay how these treatments affected camptothecin responses, non-vector-treated cells, siRNA-treated cells, and siRNA+Adp14-treated cells, were exposed to increasing doses of camptothecin as in FIG. 5B and assayed for viability 5 days post-start of vector treatment. As shown by the viability assay in FIG. 5C, reduction in ARF expression in H358 cells following exon 1β siRNA treatment, resulted in decreased sensitivity to camptothecin, while restoration and moderate overexpression of ectopic ARF slightly enhanced sensitivity, supporting the results in FIG. 5B.

Increased camptothecin sensitivity of Adp14-treated H358 cells correlated with about a 3-fold increase in ARF/topoisomerase I complex formation relative to Ad1β-treated, control siRNA treated, or non-vector treated cells, as shown by the IP/Western analysis in FIG. 5D (upper panel, lane 1 versus lanes 2-4), and with an increase in topoisomerase I activity (FIG. 5D, lower panel lane 1, bar 1 versus lanes 2-4, bars 2-4). The decreased camptothecin sensitivity of siRNA-treated H358 cells correlated with about a 3-fold decrease in ARF/topoisomerase I complex formation (FIG. 5D, upper panel lane 5), and with a decrease in topoisomerase I activity (FIG. 5D, lower panel lane 5, bar 5).

The H23 cell line, with low to undetectable levels of endogenous ARF/topoisomerase I complexes, respectively (FIG. 1B) displayed a greatly reduced response to camptothecin (FIG. 5E), consistent with studies showing that loss of topoisomerase I phosphorylation reduces activity (Pommier, et al., J Biol Chem 1990;265(16):9418-22). The fact that H23 cells cannot be sensitized to camptothecin by ectopic overexpression of ARF indicates that ARF-mediated sensitization requires its interaction with active, serine phosphorylated topoisomerase I.

### Example 7

### ARF Promotes Topoisomerase I DNA Binding.

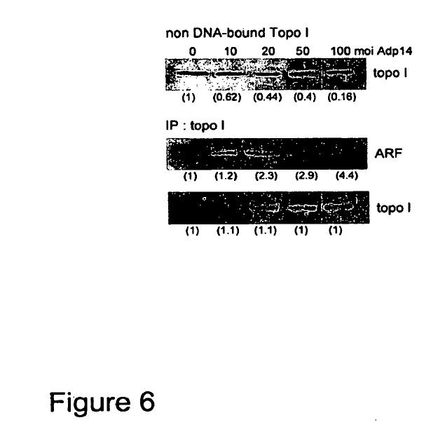

Topoisomerase I/DNA binding assay were performed to address the mechanism by which ARF activates topoisomerase I. In FIG. 6 shows the results from an immunodepletion assay in which topoisomerase I was trapped in a complex with DNA by treatment of cells with camptothecin, followed by Western analysis of nuclei prepared with NP40. Because topoisomerase I/DNA complexes are too large to enter the gel, an increase in topoisomerase I/DNA complex formation leads to a decrease in the intensity of the topoisomerase I immunoreactive band representing non-DNA-bound topoisomerase I. Treatment of H358 cells with increasing doses of Adp14 resulted in a progressive decrease in non DNA-bound topoisomerase I (FIG. 6 top panel), under conditions where co-immunoprecipitated ARF/topoisomerase I complexes, released from NP40 nuclei by DNase I treatment, increased (FIG. 4A, middle panel), and total topoisomerase I, released from NP40 nuclei by DNase I treatment, remained constant (FIG. 6, bottom panel). Thus increased ARF/topoisomerase I complex formation is accompanied by an increase in DNA bound topoisomerase I.

### Example 8

### Camptothecin Sensitivity is Dependent upon Topoisomerase I Phosphorylation and ARF Binding.

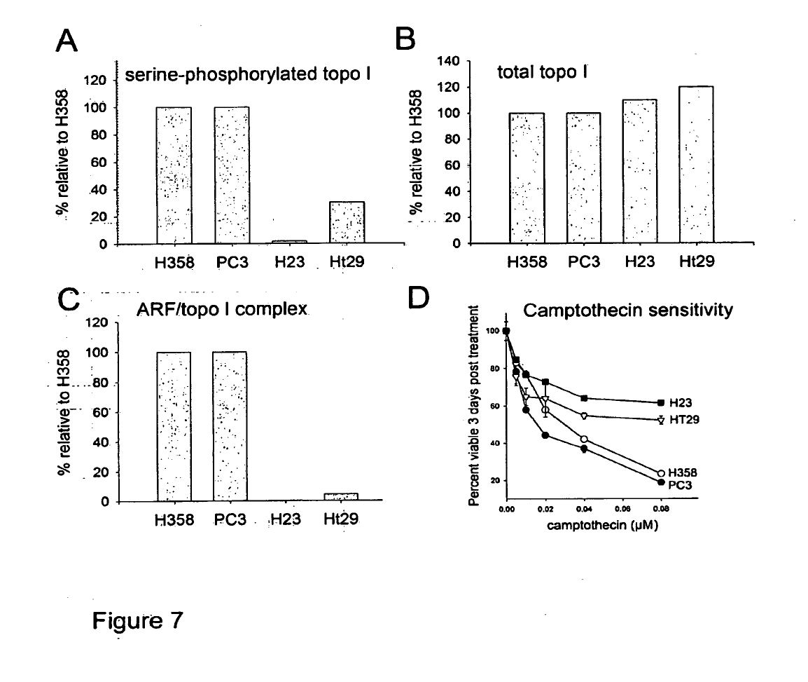

FIG. 7 shows the results of an additional topoisomerase I immunoprecipitation (IP)/Western analyses as in FIG. 3, and cell viability assays in the presence of camptothecin. This experiment was performed to confirm the relationship between topoisomerase I serine phosphorylation, ARF/topoisomerase I complex formation, and cellular camptothecin sensitivity. The Western blots were analyzed digitally and the band intensities relative to H358 are plotted as bar graphs in FIG. 7A-7C).

The PC-3 cell lines displays a level of topoisomerase I serine phosphorylation similar to H358 (FIG. 7A), a similar level of total cellular topoisomerase I (FIG. 7B), a similar level of cellular ARF/top I complex formation (FIG. 7C), and a similar degree of sensitivity to camptothecin (FIG. 7D). In contrast, both H23 and HT29 cells display a reduced level of topoisomerase I serine phosphorylation compared to H358 (FIG. 7A), although total cellular topoisomerase I is similar to that of H358 and PC-3 (FIG. 7B). H23 and HT 29 cells display reduced levels of cellular ARF/topoisomerase I complex formation (FIG. 7C), and are more resistant to camptothecin that are H358 and PC-3 cells (FIG. 7D).

### Example 9

### Hela Cells have Partially Defective ARF-topoisomerase I Complex Formation and Show Intermediate Sensitivity to Camptothecin.

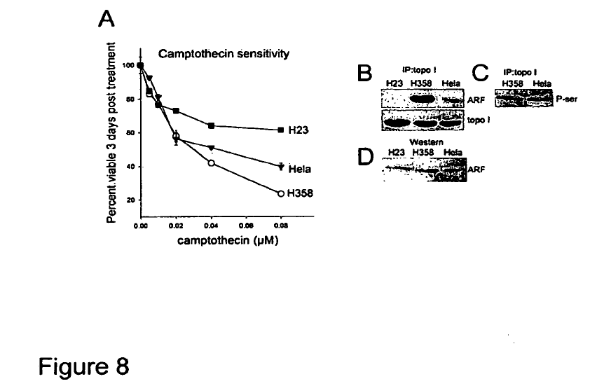

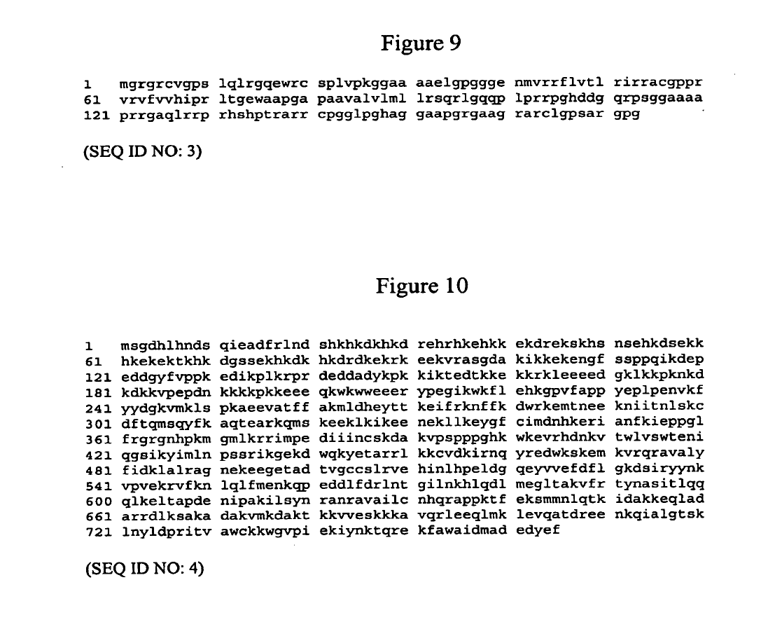

Hela cells display a sensitivity to camptothecin intermediate to that of H23 and H358 (FIG. 8A). The assay in FIG. 8A was carried out as in FIG. 7C. ARF/topoisomerase I complex formation was examined using the co-immunoprecipitation assay described for FIGS. 3A and 3C. A reduced but detectable level of ARF/topoisomerase I complex formation in Hela cells was observed, compared to H358 cells (FIG. 8B). ARF/topoisomerase I complex formation in H23 cells was undetectable (FIG. 8B), confirming previous experiments (FIG. 1C). However, topoisomerase I was serine phosphorylated in Hela cells (FIG. 8C), indicating that other factors are likely to be responsible for the failure to form ARF/topoisomerase I complexes. Total ARF levels in H23, H358, and Hela cells were found to be similar (FIG. 8D). The results indicate that defective ARF/topoisomerase I complex formation can result from cellular changes other than defective phosphorylation of topoisomerase I, and confers increased resistance to camptothecin.

### Example 10

### Treatment of Cancer in a Human

A human patient diagnosed with cancer may be treated according to the methods and principles of this disclosure. For example, a patient diagnosed with prostate cancer is administered once each day for five days, by intravenous injection, 100 moi of an adenoviral vector containing nucleic acid encoding functional CKII, operably linked to a promoter. Subsequently, the patient is administered camptothecin at 10 mg/kg/day for sixty days. This treatment regimen results in a reduction in the size of the prostate tumor, or the level of prostate-specific antigen in the blood, or both.

During the course of this treatment regimen, the prostate cancer cells contain both an elevated serine kinase biological activity (caused by infection with the CKII-containing adenoviral vector) and a topoisomerase inhibitor (i.e., camptothecin).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

1. A method for inducing apoptosis in a cell, comprising contacting said cell with an agent that increases the p14ARF-topoisomerase I complex formation and further contacting said cell with a topoisomerase I inhibitor.

2. The method of claim 1, wherein said agent increases the serine kinase biological activity in said cell.

3. The method of claim 2, wherein said agent is a vector comprising a nucleic acid encoding a serine kinase enzyme, operably linked to a promoter.

4. The method of claim 2, wherein said serine kinase phosphorylates topoisomerase I on at least one serine residue and wherein said phosphorylation is sufficient to promote p14ARF-topoisomerase I complex formation.

5. The method of claim 2, wherein said serine kinase is casein kinase II or protein kinase C.

6. The method of claim 2, wherein said serine kinase activity is increased in the nucleus of said cell.

7. The method of claim 1, wherein said agent is a vector comprising a nucleic acid encoding p14ARF or a biologically active fragment thereof, operably linked to a promoter.

8. The method of claim 7, wherein said p14ARF or biologically active fragment thereof comprises amino acid residues 66-84 of p14ARF.

9. The method of claim 1, wherein said topoisomerase I inhibitor stabilizes a topoisomerase I-DNA complex.

10. The method of claim 1, wherein said topoisomerase I inhibitor is selected from the group consisting of camptothecin, irinotecan, topotecan, and analogs thereof.

11. The method of claim 10, wherein said topoisomerase I inhibitor is camptothecin.

12. The method of claim 1, wherein said cell is a cancer cell.

13. The method of claim 12, wherein said cancer cell is selected from the group consisting of a lung cancer cell, a prostate cancer cell, a hepatocellular carcinoma cell, a breast cancer cell, a colorectal cancer cell, an acute myelogenous leukemia cell, a melanoma cell, an ovarian cancer cell, a neuroendocrine carcinoma cell, a gastric cancer cell, an esophageal cancer cell, a pancreatic cancer cell, and an adenocarcinoma cell.

14. The method of claim 13, wherein said cancer cell is present in a human patient.

15-25. (canceled)

26. A method for treating cancer in a patient, comprising administering to said patient an agent that increases the p14ARF-topoisomerase I complex formation and further administering a topoisomerase I inhibitor.

27. The method of claim 26, wherein said agent increases the serine kinase biological activity in said cell.

28. The method of claim 27, wherein said agent is a vector comprising a nucleic acid encoding a serine kinase enzyme, operably linked to a promoter.

29. The method of claim 27, wherein said serine kinase phosphorylates topoisomerase I on at least one serine residue and wherein said phosphorylation is sufficient to promote p14ARF-topoisomerase I complex formation.

30. The method of claim 27, wherein said serine kinase is casein kinase II or protein kinase C.

31. The method of claim 27, wherein said serine kinase activity is increased in the nucleus of said cell.

32. The method of claim 26, wherein said agent is a vector comprising a nucleic acid encoding p14ARF or a biologically active fragment thereof, operably linked to a promoter.

33. The method of claim 32, wherein said p14ARF or biologically active fragment thereof comprises amino acid residues 66-84 of p14ARF.

34. The method of claim 26, wherein said topoisomerase I inhibitor stabilizes a topoisomerase I-DNA complex.

35. The method of claim 26, wherein said topoisomerase I inhibitor is selected from the group consisting of camptothecin, irinotecan, topotecan, and analogs thereof.

36. The method of claim 35, wherein said topoisomerase I inhibitor is camptothecin.

37. The method of claim 26, wherein said cancer is selected from the group consisting of lung cancer, prostate cancer, hepatocellular carcinoma, breast cancer, colorectal cancer, acute myelogenous leukemia, melanoma, ovarian cancer, neuroendocrine carcinoma, gastric cancer, esophageal cancer, pancreatic cancer, and adenocarcinoma.

38-48. (canceled)

49. A method for inducing apoptosis in a cell, comprising contacting said cell with an agent that inhibits the binding of p14ARF to topoisomerase I.

50. The method of claim 49, wherein said binding is inhibited by contacting the cell with an antibody, a peptide, an aptamer, or a peptidomimetic.

51. The method of claim 49, wherein said cell is a cancer cell.

52. The method of claim 49, wherein said cell is a cancer cell is selected from the group consisting of a lung cancer cell, a prostate cancer cell, a hepatocellular carcinoma cell, a breast cancer cell, a colorectal cancer cell, an acute myelogenous leukemia cell, a melanoma cell, an ovarian cancer cell, a neuroendocrine carcinoma cell, a gastric cancer cell, an esophageal cancer cell, a pancreatic cancer cell, and an adenocarcinoma cell.

53. The method of claim 49, wherein said cell is present in a human patient.

54. The method of claim 49, wherein said p14ARF binds HDM2.

55. A method for determining the sensitivity of a cancer cell to a topoisomerase I inhibitor said method selected from the group consisting of:
(a) determining the nuclear localization of p14ARF within said cancer cell, and identifying said cancer cell as being sensitive to a topoisomerase I inhibitor when said p14ARF is substantially localized to the nucleolus and identifying a cancer cell as being resistant to a topoisomerase I inhibitor when said p14ARF is substantially disbursed in the nucleus of said cell,
(b) determining the ratio of free p14ARF to p14ARF bound to topoisomerase I in the nucleus of said cancer cell and identifying said cancer cell as being sensitive to a topoisomerase I inhibitor when said ratio is less than 1, and identifying a cancer cell as being resistant to a topoisomerase I inhibitor when said ratio is greater than 1, and
(c) determining the ratio of unphosphorylated topoisomerase I to phosphorylated topoisomerase I in the nucleus of said cancer cell, and identifying said cancer cell as being sensitive to a topoisomerase I inhibitor when said ratio is less than 1, and identifying a cancer cell as being resistant to a topoisomerase I inhibitor when said ratio is greater than 1.
